(19) United States
(12) Patent Application Publication
(10) Pub. No.: US 2023/0093507 A1
(43) Pub. Date: Mar. 23, 2023

(54) DATA PROCESSING METHOD AND APPARATUS, COMPUTER DEVICE, AND STORAGE MEDIUM

(71) Applicant: Tencent Technology (Shenzhen) Company Limited, Shenzhen (CN)

(72) Inventors: Jianguo PEI, Shenzhen (CN); Wei LIU, Shenzhen (CN); Junzhou HUANG, Shenzhen (CN)

(21) Appl. No.: 18/071,445

(22) Filed: Nov. 29, 2022

Related U.S. Application Data
(63) Continuation of application No. PCT/CN2022/071490, filed on Jan. 12, 2022.

(30) Foreign Application Priority Data
Jan. 19, 2021 (CN) 202110065836.X

(51) Int. Cl. G16B 15/30; G16B 15/20; G16B 40/20

(52) U.S. Cl. CPC G16B 15/30; G16B 15/20; G16B 40/20

(57) ABSTRACT

This application discloses a method for processing bioinformatic data performed by a computer device. The method includes: acquiring protein attribute information of a reference protein substance; generating a predicted protein fragment at the protein adjusting region in the reference protein substance by applying a protein prediction model to the protein attribute information, the protein prediction model being configured to predict a protein substance binding to a target protein substance; identifying a similar protein fragment matching the predicted protein fragment in a protein fragment database; virtually synthesizing the similar protein fragment and the reference protein substance to obtain synthetic substance auxiliary information; and the synthetic substance auxiliary information being configured to assist in generation of an antibody protein substance binding to the target protein substance.

### CROSS-REFERENCE TO RELATED APPLICATIONS

This application is a continuation application of PCT Patent Application No. PCT/CN2022/071490, entitled “DATA PROCESSING METHOD AND APPARATUS, AND COMPUTER DEVICE AND STORAGE MEDIUM” filed on Jan. 12, 2022, which claims priority to Chinese Patent Application No. 202110065836.X, filed with the State Intellectual Property Office of the People's Republic of China on Jan. 19, 2021, and entitled “DATA PROCESSING METHOD AND APPARATUS, COMPUTER DEVICE, AND STORAGE MEDIUM”, all of which are incorporated herein by reference in their entirety.

### FIELD OF THE TECHNOLOGY

This application relates to the technical field of artificial intelligence, in particular to a data processing technology.

### BACKGROUND OF THE DISCLOSURE

A target protein is a pathogenic protein, and when an antibody protein configured to bind to the target protein is designed, a reference antibody protein originally existing in a human body is generally required to be modified, and the modified reference antibody protein is the antibody protein configured to bind to the target protein.

For specific implementation, the reference antibody protein may be modified by using short chain polypeptides in a short chain polypeptide database, where the short chain polypeptide database generally includes a plurality of short chain polypeptides collected from nature, and one short chain polypeptide is one protein fragment. In the related art, generally the short chain polypeptides may be randomly selected from the short chain polypeptide database continuously, to modify the reference antibody protein, and each time the reference antibody protein is modified by using one short chain polypeptide, it is necessary to evaluate whether the modified reference antibody protein meets the standard of binding to the target protein needs. Until it is evaluated that the modified reference antibody protein meets the standard of binding to the target protein, the selection of the short chain polypeptides from the short chain polypeptide database to modify the reference antibody protein can be stopped.

Therefore, in the related art, randomness of selecting the short chain polypeptides configured to modify the reference antibody protein is very high, and this will lead to that the reference antibody protein needs to be modified by a large amount of short chain polypeptides in many cases to obtain the reference antibody protein meeting the standard of binding to the target protein, that is, the efficiency of acquiring the antibody protein configured to bind to the target protein is low.

### SUMMARY

This application provides a data processing method and apparatus, a computer device and a storage medium, which can improve the efficiency of acquiring antibody protein.

An aspect of this application provides a bioinformatic data processing method, performed by the computer device, the method including:

acquiring protein attribute information of a reference protein substance; the reference protein substance including a protein adjusting region;

generating a predicted protein fragment at the protein adjusting region in the reference protein substance by applying a protein prediction model to the protein attribute information, the protein prediction model being configured to predict a protein substance binding to a target protein substance;

identifying a similar protein fragment matching the predicted protein fragment in a protein fragment database; and

virtually synthesizing the similar protein fragment and the reference protein substance to obtain synthetic substance auxiliary information; and the synthetic substance auxiliary information being configured to assist in generation of an antibody protein substance binding to the target protein substance.

Another aspect of this application provides a data processing apparatus, including:

an attribute acquiring module, configured to acquire the protein attribute information of the reference protein substance; the reference protein substance including a protein adjusting region;

a predicted fragment generating module, configured to generate a predicted protein fragment at the protein adjusting region in the reference protein substance by applying a protein prediction model to the protein attribute information, the protein prediction model being configured to predict a protein substance binding to a target protein substance;

a fragment matching module, configured to identify a similar protein fragment matching the predicted protein fragment in a protein fragment database; and

a substance synthesizing module, configured to virtually synthesize the similar protein fragment and the reference protein substance to obtain synthetic substance auxiliary information; and the synthetic substance auxiliary information being configured to assist in generation of an antibody protein substance binding to the target protein substance.

An aspect of this application provides a computer device, including a memory and a processor, the memory storing a computer program; and the computer program, when executed by the processor, causing the computer device to perform the bioinformatic data processing method according to the aspect of this application.

An aspect of this application provides a non-transitory computer-readable storage medium storing a computer program, the computer program including a program instruction, the program instruction, when executed by a processor of a computer device, causing the computer device to perform the foregoing bioinformatic data processing method according to the foregoing aspect.

An aspect of this application provides a computer program product or a computer program, the computer program product or the computer program including computer instructions, the computer instructions being stored in a computer-readable storage medium. A processor of a computer device reads the computer instructions from the computer-readable storage medium, and executes the computer instructions, so that the computer device performs the method provided in the various implementations in the foregoing aspect.

In this application, the protein attribute information of the reference protein substance may be acquired; the reference protein substance including a protein adjusting region; generating a predicted protein fragment at the protein adjusting region in the reference protein substance by using a protein prediction model according to the protein attribute information; the protein prediction model being obtained on the basis of training of the target protein substance; the protein prediction model being configured to predict a protein substance binding to the target protein substance; matching a similar protein fragment of the predicted protein fragment in a protein fragment database; virtually synthesizing the similar protein fragment and the reference protein substance to obtain synthetic substance auxiliary information; and the synthetic substance auxiliary information being configured to assist in generation of an antibody protein substance binding to the target protein substance. Therefore, according to the method provided by this application, on the basis of the predicted protein fragment obtained by prediction of the protein prediction model, the similar protein fragment configured to modify the protein fragment at the protein adjusting region in the reference protein substance is rapidly matched, then the antibody protein substance configured to bind to the target protein substance may be rapidly generated on the basis of the similar protein fragment, and thus, the efficiency of acquiring the antibody protein substance is improved.

### BRIEF DESCRIPTION OF THE DRAWINGS

FIG. 1 is a schematic structural diagram of a network architecture according to an embodiment of this application.

FIG. 2 is a schematic diagram of a scenario of data prediction according to this application.

FIG. 3 is a schematic flowchart of a data processing method according to this application.

FIG. 4 is a schematic diagram of a scenario of fragment prediction according to this application.

FIG. 5 is a schematic diagram of a scenario of fragment generation according to this application.

FIG. 6 is a schematic diagram of a scenario of model training according to this application.

FIG. 7 is a schematic diagram of a scenario of synthesis of an antibody protein substance according to this application.

FIG. 8 is a schematic diagram of a scenario of data interaction according to this application.

FIG. 9 is a schematic structural diagram of a data processing apparatus according to this application.

FIG. 10 is a schematic structural diagram of a computer device according to this application.

### DESCRIPTION OF EMBODIMENTS

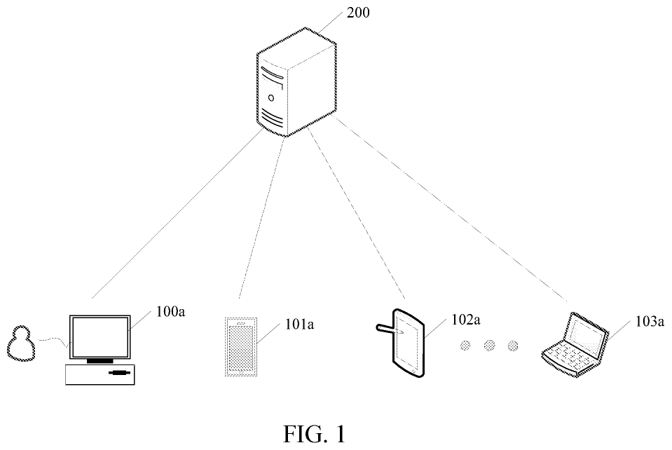

Referring to FIG. 1, FIG. 1 is a schematic structural diagram of a network architecture according to an embodiment of this application. As shown in FIG. 1, the network architecture may include a server 200 and a terminal device cluster, and the terminal device cluster may include one or more terminal devices, and the number of the terminal devices is not limited here. As shown in FIG. 1, the plurality of terminal devices may specifically include a terminal device 100a, a terminal device 101a, a terminal device 102a . . . and a terminal device 103a. As shown in FIG. 1, the terminal device 100a, the terminal device 101a, the terminal device 102a . . . and the terminal device 103a may all be in network connection with the server 200, so that each terminal device is in data interaction with the server 200 by using network connection.

The server 200 shown in FIG. 1 may be an independent physical server, or may be a server cluster including a plurality of physical servers or a distributed system, or may be a cloud server providing basic cloud computing services, such as a cloud service, a cloud database, cloud computing, a cloud function, cloud storage, a network service, cloud communication, a middleware service, a domain name service, a security service, a content delivery network (CDN), big data an artificial intelligence platform. The terminal devices may be: an intelligent terminal such as a smart phone, a tablet computer, a notebook computer, a desktop computer and an intelligent television. The following takes communication between the terminal device 100a and the server 200 as an example to specifically describe the embodiment of this application.

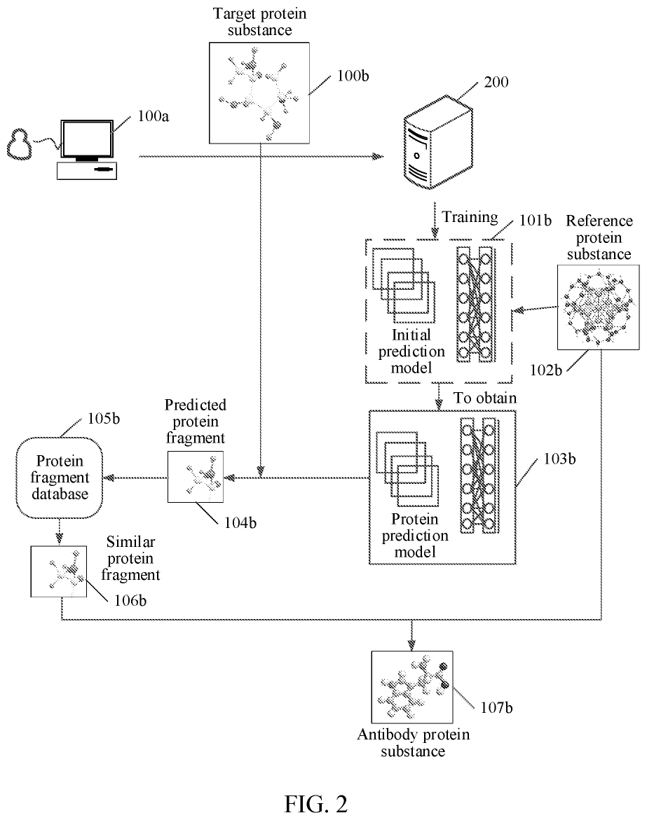

Referring to FIG. 2 together, FIG. 2 is a schematic diagram of a scenario of data prediction according to this application. Referring to FIG. 2, the terminal device 100a may be a pharmaceutical-factory-oriented terminal device. The terminal device 100a may provide a target protein substance 100b for the server 200, for example, the terminal device 100a may transmit related description information of the target protein substance 100b to the server 200. The related description information of the target protein substance 100b is description information used for determining the target protein substance 100b uniquely, for example, the related description information may be structure information, torsion angle information, protein sequence information and the like of the target protein substance 100b. The target protein substance 100b is a pathogenic protein, for example, the target protein substance 100b may be a cancer-diseased protein in a human body.

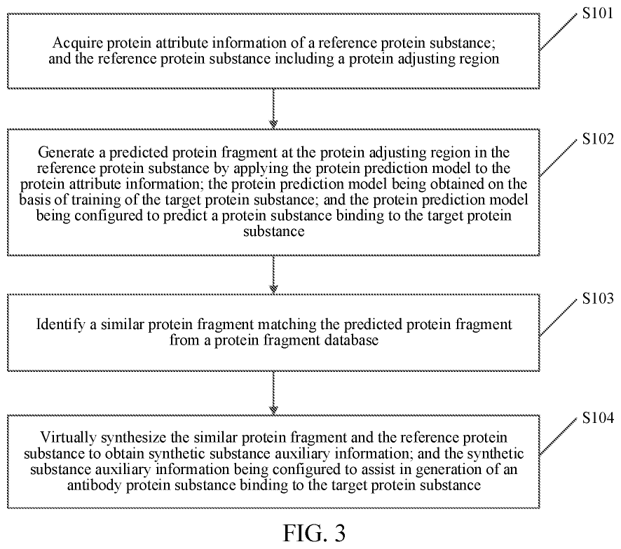

After the server 200 acquires the target protein substance 100b provided by the terminal device 100a, the server 200 may train the initial prediction model 101b on the basis of the target protein substance 100b and the reference protein substance 102b jointly so as to obtain the protein prediction model 103b. The reference protein substance 102b is a protein substance existing in a human body and capable of binding to the target protein substance 100b after being modified. The protein prediction model 103b obtained by training the initial prediction model 101b is configured to predict a protein substance capable of binding to the target protein substance 100b. By predicting the protein substance binding to the target protein substance, an antibody protein substance configured to treat a disease (such as cancer) to which the target protein substance 100b belongs. The specific process of training the initial prediction model 101b to obtain the protein prediction model 103b may be referred to the following related description in the corresponding embodiment of FIG. 3.

The reference protein substance 102b may include a protein adjusting region, and the protein adjusting region is a region capable of being modified in the reference protein substance 102b. A protein fragment 104b of the reference protein substance 102b at the protein adjusting region may be generated by using the protein prediction model 103b, and the protein fragment 104b may be referred to as a predicted protein fragment, and the protein fragment 104b is the modified form of the protein fragment of the reference protein substance 102b at the protein adjusting region.

A protein fragment database 105b may include a plurality of protein fragments collected from the nature, and the server 200 may match a similar protein fragment 106b of the predicted protein fragment 104b in the protein fragment database 105b. Then, the server 200 may synthesize the similar protein fragment 106b and the reference antibody protein 102b, and an antibody protein substance 107b may be obtained according to the synthesized result. The antibody protein substance is configured to bind to the target protein substance 100b to achieve the purpose of treating a disease (such as cancer) to which the target protein substance 100b belongs. The specific process of acquiring the antibody protein substance 107b may also be referred to the following related description in an embodiment corresponding to FIG. 3.

By the method provided by this application, AI may be applied to a medical pharmacy scenario for assisting in medical pharmacy, matching efficiency of a similar protein fragment is improved, and the efficiency of acquiring an antibody protein substance for a target protein substance may be improved. Therefore, by the method provided by this application, the medical pharmacy expense may be saved, and the medical pharmacy speed is increased.

Referring to FIG. 3, FIG. 3 is a schematic flowchart of a data processing method according to this application, as shown in FIG. 3, the method may include:

Step S101: Acquire protein attribute information of a reference protein substance; and the reference protein substance including a protein adjusting region.

Specifically, the execution subject of the embodiment of this application may be one computer device or a computer device cluster composed of a plurality of computer devices. The computer device may be a server, and may also be a terminal device. Therefore, the method provided by this embodiment of this application may be performed by the server, or may be performed by the terminal device, or may be performed by both the server and the terminal device. The following specifically describes embodiments of this application by taking an execution subject as a server.

The reference protein substance is a certain specific protein which exists in a human body and can be modified, a macromolecular antibody protein may be obtained by modifying the reference protein substance, the macromolecular antibody protein is a protein for treating diseases, and the macromolecular antibody protein may bind to a pathogenic protein to achieve the effect of treating the diseases. The reference antibody protein may be a TCL protein (human recombinant protein) existing in a human body.

In general, the reference protein substance may include a plurality of (at least two) amino acids, and the server may acquire amino acid structure information of each amino acid included in the reference protein substance and acquire amino acid torsion angle information of each amino acid included in the reference protein substance.

The amino acid structure information of amino acids may be secondary structure information of the amino acids, the secondary structure of the amino acids includes four types including α-helix, β-sheet, β-turn and random coil, and correspondingly, the amino acid structure information of the amino acids may be the secondary structure information of the amino acids in the α-helix, β-sheet, β-turn and random coil. The secondary structure information of the amino acids is the secondary structure information of a protein substance to which the amino acids belong, and the secondary structure information is specific conformation formed by coiling or folding polypeptide main chain skeleton atoms in the protein substance along a certain axis, namely the spatial position arrangement of the main chain skeleton atoms of a peptide chain.

The amino acid torsion angle information of the amino acids is the torsion angle information of the reference protein substance. The torsion angle information of a protein substance includes a torsion angle of the protein substance, and the torsion angle is an angle formed by crossing other bonds on adjacent carbons when a single bond in the protein substance rotates. The amino acid torsion angle information of one amino acid may include 3 angles, and the 3 angles are Phi angle, Psi angle and Omega angle respectively. Specifically, the phi angle is an angle rotated around a bond N—Ca (a chemical bond), the psi angle is an angle rotated around a bond Ca—C(a chemical bond), and the omega angle is an angle rotated around a C—N bond (a chemical bond).

An amino acid in the reference protein substance may have one piece of amino acid structure information and one piece of amino acid torsion angle information, and the server may acquire the amino acid structure information and the amino acid torsion angle information of all amino acids in the reference protein substance as protein attribute information of the reference protein substance. In addition, the protein attribute information may further include a protein sequence (which may be simply referred to as proteins) of the reference protein substance, and the protein sequence is a computer representation of the reference protein substance, that is, a machine language of the reference protein substance. The amino acid structure information and the amino terminal torsion angle information, included in the reference protein substance, of the amino acids can represent the structure information and the torsion angle information of the reference protein substance.

The reference protein substance is a reference antibody protein, the reference protein substance may include a variable region (CDR), and the variable region is also a region, which may be modified, in the reference protein substance. A protein at a diseased portion in a pathogenic protein may be referred to as a target protein, the target protein is a protein, and the target protein may also be referred to as a target protein substance. The target protein substance is a protein which is diseased in a human body, and the generated macromolecular antibody protein needs to bind to the target protein substance so as to achieve the purpose of treating human diseases.

It is understood that there are generally different target protein substances for different diseases, and that different diseases may generally correspond to different variable regions (namely modified regions) in the reference protein substance. In other words, with regard to different target protein substances, the reference protein substances of the target protein substances may be the same, but the different target protein substances may correspond to different variable regions in the same reference protein substance.

In this embodiment of this application, the target protein substance binding to the generated macromolecular antibody protein is required, may be any paraprotein in a human body, and the specific disease type of the target protein substance may be determined according to an actual application scenario, which is not limited herein, and is herein collectively referred to as the target protein substance, and a process of generating the macromolecular antibody protein of the target protein substance is described herein as an example.

The target protein substance may be provided by a third-party device, and the third-party device may be pharmaceutical-factory-oriented. When a pharmaceutical factory needs to generate a macromolecular antibody protein for binding to a certain target protein substance, the pharmaceutical factory may provide the target protein substance to the server by using the third-party device, for example, the pharmaceutical factory may transmit the related description information of the target protein substance to the server by using the third-party device, the related description information is information for describing and determining the target protein substance, for example, the information may include protein sequence information and protein structure information of the target protein substance, and the like. Or, the pharmaceutical factory may provide a pdb file of the target protein substance to the server by using the third-party device, where the pdb file is a program data file, and the pdb file of the target protein substance may visualize a three-dimensional picture of the target protein substance, that is, may present a space picture of the target protein substance.

After acquiring the target protein substance provided by the third-party device, the server may identify a target protein type of the target protein substance further, where the target protein type can represent a type of a disease to which the target protein substance belongs, such as a type of cancer or a type of virus. A mapping relationship between the disease types and the corresponding variable regions in the reference protein substance is maintained on the server, and the mapping relationship indicates the corresponding variable region in the reference protein substance for each disease type.

Based on this, the server may search a variable region which has a mapping relationship with the target protein type in the reference protein substance according to the identified target protein type of the target protein substance, and then the variable region which corresponds to the target protein substance in the searched reference protein substance is used as a protein adjusting region in the reference protein substance.

Step S102: Generate a predicted protein fragment at the protein adjusting region in the reference protein substance by applying the protein prediction model to the protein attribute information. The protein prediction model being obtained on the basis of training of the target protein substance; and the protein prediction model being configured to predict a protein substance binding to the target protein substance.

Specifically, the server may call the protein prediction model, the protein prediction model is obtained by training of the target protein substance, and the protein prediction model is configured to predict the protein substance binding to the target protein substance. The server may input the acquired protein attribute information of the reference protein substance into the protein prediction model, and then the protein prediction model may correspondingly generate the predicted protein fragment at the protein adjusting region in the reference protein substance. The predicted protein fragment is a modified form of the protein fragment at the protein adjusting region in the reference protein substance. A protein fragment may be a protein fragment composed of a plurality of amino acids connected to one another.

Optionally, the server may input the whole protein attribute information of the reference protein substance into the protein prediction model so as to generate the predicted protein fragment in the protein prediction model. Or, the server may also input the protein attribute information at the protein adjusting region in the reference protein substance into the protein prediction model so as to generate the predicted protein fragment in the protein prediction model. The protein attribute information at the protein adjusting region in the reference protein substance may include the amino acid structure information and the amino acid torsion angle information of the amino acids of the reference protein substance at the protein adjusting region, and may also include protein sequence information of a protein fragment of the reference protein fragment at the protein adjusting region. In a specific implementation, the whole protein attribute information of the reference protein substance may be selected and inputted into the protein prediction model according to the actual application scenario, or only the protein attribute information at the protein adjusting region in the reference protein substance can be inputted into the protein prediction model.

A process of generating the predicted protein fragment at the protein adjusting region in the reference protein substance by applying the protein prediction model to the protein attribute information may be that:

amino acids at the protein adjusting region in the reference protein substance are determined as adjusted amino acids, and the adjusted amino acids may be plural. The protein prediction model may generate predicted structure information and predicted torsion angle information of each adjusted amino acid according to the inputted protein attribute information of the reference protein substance, one adjusted amino acid may correspond to one piece of predicted structure information and one piece of predicted torsion angle information, one piece of predicted structure information may be structure information of any one secondary structure of four secondary structures of α-helix, β-sheet, β-turn and random coil, and one piece of predicted torsion angle information may include 3 angles including Phi angle, Psi angle and Omega angle.

Further, a specific process of predicting the predicted structure information of each adjusted amino acid in the protein prediction model and generating the predicted structure information of the adjusted amino acids in the protein prediction model may be that:

since a secondary structure of an amino acid includes α-helix, β-sheet, β-turn, and random coil, one secondary structure may be understood as one structure dimension (which may be referred to as an amino acid structure dimension) of an amino acid. Thus, the amino acid structure dimensions of an amino acid may include four amino acid structure dimensions of α-helix, β-sheet, β-turn and random coil.

Based on this, the protein prediction model may predict the sampling probability of the adjusted amino acid on each amino acid structure dimension. With regard to one adjusted amino acid, one amino acid structure dimension may correspond to one sampling probability, and the sampling probability can represent the sampling probability of the predicted secondary structure of the adjusted amino acid on the corresponding amino acid structure dimension. The amino acid structure dimension with the maximum sampling probability in all amino acid structure dimensions may be taken as a target structure dimension. One adjusted amino acid corresponds to one target structure dimension.

Then, the server may sample structure parameters on the target structure dimension, predicted structure information corresponding to the adjusted amino acid is generated, and the generated predicted structure information is the generated secondary structure of the adjusted amino acid on the target structure dimension. For example, if the target structure dimension is the structure dimension of α-helix, and then the generated predicted structure information corresponding to the adjusted amino acid is α-helix secondary structure of the adjusted amino acid. It is understood that a process of sampling the structure parameters is a process of generating the predicted structure information.

Further, a specific process of predicting the predicted torsion angle information of each adjusted amino acid in the protein prediction model and generating the predicted torsion angle information of the adjusted amino acid in the protein prediction model may be that:

since the torsion angle of an amino acid may include Phi angle, Psi angle and Omega angle, the Phi angle, Psi angle and Omega angle may range from 0 to 360 degrees, every 10 degrees may be a sampling interval, and for example, [0, 10] may be a sampling interval. Therefore, each of the Phi angle, Psi angle and Omega angle corresponds to 36 (namely 360/10) sampling intervals, one sampling interval may be understood as one torsion angle dimension (which may be referred to as an amino acid torsion angle dimension), and thus, the torsion angle of an amino acid has 36*36*36 amino acid torsion angle dimensions in total.

For example, if one sampling interval of the Phi angle is [0, 10], one sampling interval of the Psi angle is [10, 20] and one sampling interval of the Omega angle is [20, 30], then one amino acid torsion angle dimension may be Phi [0, 10]-Psi [10, 20]-Omega [20, 30], the amino acid torsion angle dimension represents that the Phi angle ranges from 0 to 10 degrees, the Psi angle ranges from 10 to 20 degrees, and the Omega angle ranges from 20 to 30 degrees.

Optionally, the angle range of one sampling interval is not limited to 10 degrees, and may also be other angle ranges, and the value of the angle range of one sampling interval is determined according to the actual application scenario, which is not limited to this. For example, one sampling interval may further be 5 degrees, and for example, [0, 5] may be a sampling interval. At the moment, each of the Phi angle, the Psi angle and the Omega angle may correspond to 72 (namely 360/5) sampling intervals, and thus, the torsion angle of an amino acid has 72*72*72 amino acid torsion angle dimensions in total.

For example, if one sampling interval of the Phi angle is [0, 5], one sampling interval of the Psi angle is [5, 10] and one sampling interval of the Omega angle is [10, 15], then one amino acid torsion angle dimension may be [0, 5]-[5, 10]-[10, 15], and the amino acid torsion angle dimension represents that the Phi angle ranges from 0 to 5 degrees, the Psi angle ranges from 5 to 10 degrees, and the Omega angle ranges from 10 to 15 degrees.

Based on this, the protein prediction model may predict the sampling probability of the adjusted amino acid on each amino acid torsion angle dimension. With regard to one adjusted amino acid, one amino acid torsion angle dimension may correspond to one sampling probability, and the sampling probability can represent the probability of the predicted torsion angle of the adjusted amino acid on the corresponding amino acid torsion angle dimension. The amino acid torsion angle dimension with the maximum sampling probability in all the amino acid torsion angle dimensions may be taken as a target torsion angle dimension. One adjusted amino acid corresponds to one target torsion angle dimension.

Then, the server may sample torsion angle parameters on the target torsion angle dimensions, predicted torsion angle information corresponding to the adjusted amino acid is generated, and the generated predicted torsion angle information is a generated torsion angle of the adjusted amino acid on the target torsion angle dimension. For example, torsion angle parameters may be sampled by using an intermediate angle, for example, one sampling interval may be 10 degrees, the sampling interval of the 10 degrees may be [0, 10], and then the angle obtained by sampling in the sampling interval may be 5 degrees.

For example, if one amino acid torsion angle dimension is Phi [0, 10]-Psi[10, 20]-Omega [20, 30], then the Phi angle obtained by sampling in [0, 10] may be 5 degrees, the Psi angle obtained by sampling in [10, 20] may be 15 degrees, and the Omega angle obtained by sampling in [20, 30] may be 25 degrees, and correspondingly, the predicted torsion angle information, which is generated finally by sampling, of the adjusted amino acid is a Phi angle of 5 degrees, a Psi angle of 15 degrees and an Omega angle of 25 degrees. It is understood that a process of sampling the torsion angle parameters is a process of generating the predicted torsion angle information.

By the above-mentioned process, predicted structure information and predicted torsion angle information which correspond to each adjusted amino acid are generated on the basis of the predicted structure information and the predicted torsion angle information which correspond to each adjusted amino acid, the predicted protein fragment at the protein adjusting region may be determined, the protein fragment may be referred to as a predicted protein fragment, the structure information of the predicted protein fragment is the predicted structure information of the adjusted amino acid, and the torsion angle information of the predicted protein fragment is the predicted torsion angle information of the adjusted amino acid.

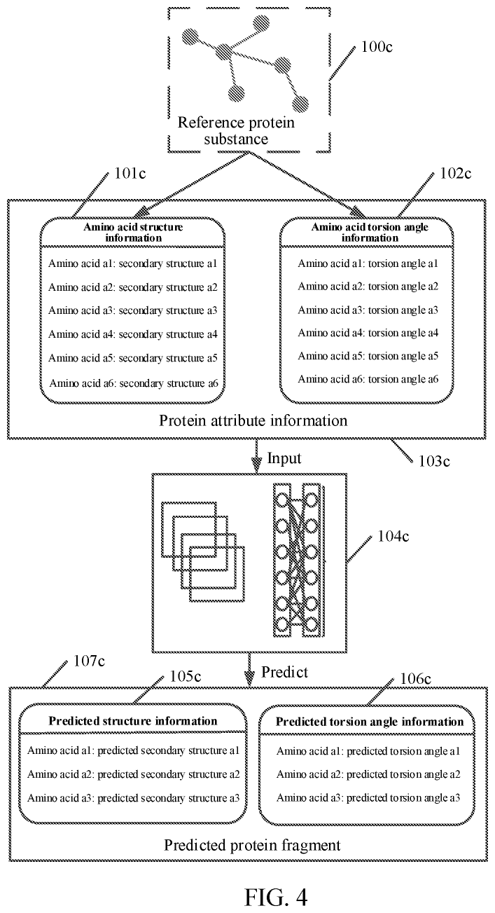

Referring to FIG. 4, FIG. 4 is a schematic diagram of a scenario of fragment prediction according to this application. As shown in FIG. 4, it is assumed that the reference protein substance 100c includes 6 amino acids in total, and the 6 amino acids are respectively an amino acid a1, an amino acid a2, an amino acid a3, an amino acid a4, an amino acid a5 and an amino acid a6. It is assumed that amino acids at the protein adjusting region in the reference protein substance 100c include an amino acid a1, an amino acid a2 and an amino acid a3, namely adjusted amino acids in the reference protein substance 100c include an amino acid a1, an amino acid a2 and an amino acid a3.

The amino acid structure information for each amino acid in the reference protein substance 100c may be the actual secondary structure of each amino acid, as shown in Box 101c, a secondary structure a1 for the amino acid a1, a secondary structure a2 for the amino acid a2, a secondary structure a3 for the amino acid a3, a secondary structure a4 for the amino acid a4, a secondary structure a5 for the amino acid a5 and a secondary structure a6 for the amino acid a6 are included.

Amino acid torsion angle information of each amino acid in the reference protein substance 100c may be a real torsion angle of each amino acid, as shown in Box 102c, a torsion angle a1 of the amino acid a1, a torsion angle a2 of the amino acid a2, a torsion angle a3 of the amino acid a3, a torsion angle a4 of the amino acid a4, a torsion angle a5 of the amino acid a5 and a torsion angle a6 of the amino acid a6 are included.

Therefore, by using a secondary structure of each amino acid in Box 101c above-mentioned and a torsion angle of each amino acid in Box 102c, protein attribute information 103c of the reference protein substance 100c may be obtained.

The server may input the protein attribute information 103c into the protein prediction model 104c, and the predicted structure information and the predicted torsion angle information of each adjusted amino acid in the reference protein substance 100c may be generated in the protein prediction model 104c. The predicted structure information of each adjusted amino acid may be a predicted secondary structure of each adjusted amino acid, and the predicted secondary structure is a predicted secondary structure of the adjusted amino acid of the protein prediction model 104c. As shown in Box 105c, the predicted structure information of each adjusted amino acid includes a predicted secondary structure a1 of the amino acid a1, a predicted secondary structure a2 of the amino acid a2 and a predicted secondary structure a3 of the amino acid a3.

The predicted torsion angle information of each amino acid may be the predicted torsion angle of each adjusted amino acid, and the predicted torsion angle is the predicted torsion angle of the adjusted amino acid of the protein prediction model. As shown in Box 106c, the predicted torsion angle information of each adjusted amino acid includes a predicted torsion angle a1 of the amino acid a1, a predicted torsion angle a2 of the amino acid a2 and a predicted torsion angle a3 of the amino acid a3.

Therefore, on the basis of the predicted structure information of each adjusted amino acid in Box 107c above-mentioned and the predicted torsion angle information of each adjusted amino acid in Box 106c, a predicted protein fragment 107c may be obtained.

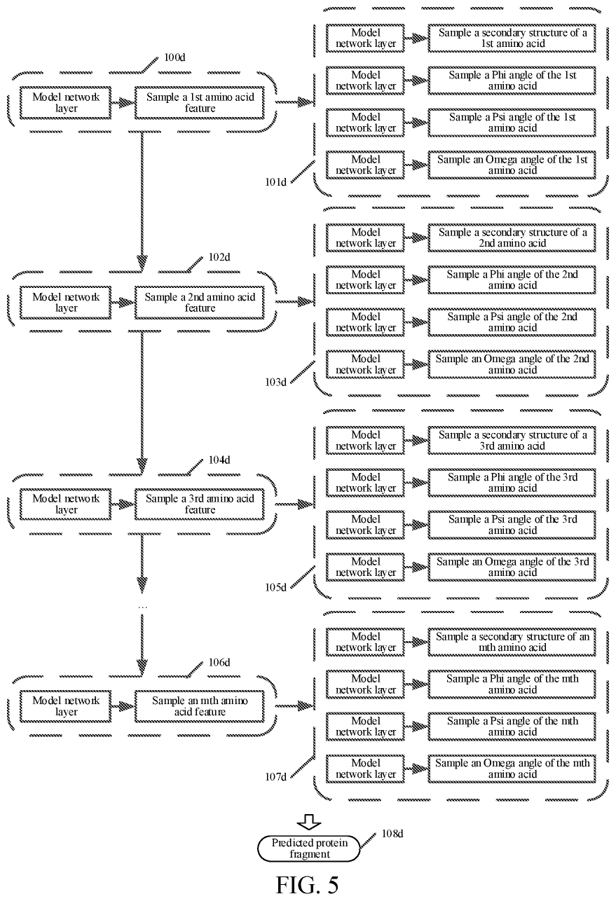

Referring to FIG. 5, FIG. 5 is a schematic diagram of a scenario of fragment generation according to this application. As shown in FIG. 5, a 1st amino acid to an mth amino acid may be adjusted amino acids of the reference protein substance at the protein adjusting region. When the predicted protein fragment is generated by using the protein prediction model, the protein prediction model may sample the predicted structure information and the predicted torsion angle information of each adjusted amino acid by using a model network layer in the protein prediction model.

As shown in Box 100d, the protein prediction model may sample an amino acid feature of the 1st amino acid, as shown in Box 101d, sampling the amino acid feature of the 1st amino acid includes sampling the secondary structure of the 1st amino acid, sampling the Phi angle of the 1st amino acid, sampling the Psi angle of the 1st amino acid and sampling the Omega angle of the 1st amino acid. The secondary structure, obtained by sampling, of the 1st amino acid is the predicted structure information of the 1st amino acid, and the Phi angle, Psi angle and Omega angle, which are obtained by sampling, of the 1st amino acid are the predicted torsion angle information of the 1st amino acid.

As shown in Box 102d, the protein prediction model may sample an amino acid feature of a 2nd amino acid, as shown in Box 103d, sampling the amino acid feature of the 2nd amino acid includes sampling a secondary structure of the 2nd amino acid, sampling a Phi angle of the 2nd amino acid, sampling a Psi angle of the 2nd amino acid and sampling an Omega angle of the 2nd amino acid. The secondary structure, obtained by sampling, of the 2nd amino acid is the predicted structure information of the 2nd amino acid, and the Phi angle, Psi angle and Omega angle, which are obtained by sampling, of the 2nd amino acid are the predicted torsion angle information of the 2nd amino acid.

As shown in Box 104d, the protein prediction model may sample an amino acid feature of a 3rd amino acid, as shown in Box 105d, sampling the amino acid feature of the 3rd amino acid includes sampling a secondary structure of the 3rd amino acid, sampling a Phi angle of the 3rd amino acid, sampling a Psi angle of the 3rd amino acid and sampling an Omega angle of the 3rd amino acid. The secondary structure, obtained by sampling, of the 3rd amino acid is the predicted structure information of the 3rd amino acid, and the Phi angle, Psi angle and Omega angle, which are obtained by sampling, of the 3rd amino acid are the predicted torsion angle information of the 3rd amino acid.

By the above-mentioned process, the protein prediction model may sample the amino acid feature of each adjusted amino acid until the amino acid feature of the last adjusted amino acid (for example, the mth amino acid) is obtained by sampling.

As shown in Box 106d, the protein prediction model may sample the amino acid feature of the mth amino acid, as shown in Box 107d, sampling the amino acid feature of the mth amino acid includes sampling a secondary structure of the mth amino acid, sampling a Phi angle of the mth amino acid, sampling a Psi angle of the mth amino acid and sampling an Omega angle of the mth amino acid. The secondary structure, obtained by sampling, of the mth amino acid is the predicted structure information of the mth amino acid, and the Phi angle, Psi angle and Omega angle, which are obtained by sampling, of the mth amino acid are the predicted torsion angle information of the mth amino acid.

Then, the protein prediction model may generate a predicted protein fragment 108d by using the amino acid feature, obtained by sampling above-mentioned, of each adjusted amino acid (including the 1st amino acid to the mth amino acid).

How to train the protein prediction model above-mentioned is described in detail below.

In an embodiment of this application, the protein prediction model is trained by a reinforcement learning method, training data and prediction data of the protein prediction model are generally the same, and therefore, it is understood that the training process of the protein prediction model is equivalent to a process of continuously updating the predicted protein fragment.

In other words, each time the server acquires a target protein substance, the protein prediction model may be trained on the basis of the target protein substance and the reference protein substance, and the predicted protein fragment at the protein adjusting region in the reference protein substance is predicted in real time by using the protein prediction model obtained by training, and the predicted protein fragment is configured to assist in generation of a macromolecular antibody protein binding to the target protein substance.

In this embodiment of this application, the untrained protein prediction model may be referred to as an initial prediction model. In other words, the protein prediction model may be obtained by training the initial prediction model, the initial prediction model may be a deep neural network, for example, the initial prediction model may be an RNN network (recurrent neural network), an LSTM network (long short term memory network) or other neural network structures.

Similarly, since the training data is the target protein substance and the reference protein substance above-mentioned, the protein attribute information of the reference protein substance may be inputted into the initial prediction model, and a protein fragment at the protein adjusting region in the reference protein substance is predicted and generated by using the initial prediction model, the protein fragment, which is predicted and obtained by the initial prediction model, at the protein adjusting region in the reference protein substance may be referred to as a sample predicted protein fragment, and a principle of generating the sample predicted protein fragment by the initial prediction model is the same as a principle of generating the predicted protein fragment by the protein prediction model, but the model parameters of the initial prediction model are different from the model parameters of the protein prediction model, so that the sample predicted protein fragment generated by the initial prediction model and the predicted protein fragment generated by the protein prediction model are different.

Further, a protein fragment database is further maintained in the server, the protein fragment database may include a plurality of protein fragments, one protein fragment is a short chain polypeptide, one short chain polypeptide may be composed of a plurality of amino acids, and therefore, the protein fragment database may also be referred to as a short chain polypeptide database. It is understood that, the protein fragment database may include all the short chain polypeptides which are collected historically and exist in nature, and more specifically, the protein fragment database may also include protein fragments obtained by splitting and scattering proteins collected historically.

After the sample predicted protein fragment generated by the initial prediction model is acquired, the server may match a protein fragment similar to the sample predicted protein fragment in the protein fragment database, and the matched protein fragment similar to the sample predicted protein fragment may be referred to as a sample similar protein fragment. The length of the matched sample similar protein fragment is the same as the length of the sample predicted protein fragment, and the length of the sample predicted protein fragment is the same as the length of the protein fragment at the protein adjusting region in the reference protein substance. The number of amino acids may be used for measuring the length of protein fragments, and protein fragments with the same number of amino acids may be considered to have the same length. A sample similar protein fragment matched with the sample predicted protein fragment may be searched in the protein fragment database by using a FragmentPicker tool (fragment selecting tool).

Then, the matched sample similar protein fragment and the reference protein substance may be virtually synthesized to obtain sample synthetic substance auxiliary information. The virtual synthesis of the sample similar protein fragment and the reference protein substance may be performed by replacing the protein fragment at the protein adjusting region in the reference protein substance with the sample similar protein fragment, and correspondingly, the sample synthetic substance auxiliary information may be related description information (for example, description information that can describe and uniquely determine the protein substance, such as protein structure information, protein sequence information and protein torsion angle information) of the protein substance obtained by synthesizing the sample similar protein fragment and the reference protein substance, that is, related description information of a new protein substance obtained by replacing the protein fragment at the protein adjusting region in the reference protein substance with the sample similar protein fragment.

It is understood that, the sample synthetic substance auxiliary information above-mentioned is used for assisting in generation of a sample antibody protein substance binding to the target protein substance, the sample antibody protein substance is a protein substance obtained by replacing the protein fragment at the protein adjusting region in the reference protein substance with a sample similar protein fragment, and the sample antibody protein substance is a macromolecular antibody protein which is generated on the basis of the matched sample similar protein fragment and is configured to bind to a target protein substance.

Then, the server may further acquire binding strength between the sample antibody protein substance and the target protein substance, and the binding strength may be referred to as sample binding strength. As the name implies, the sample binding strength is the strength of binding of reaction between the sample antibody protein fragment substance and the target protein substance.

Along with increasing of the binding strength between the antibody protein substance and the target protein substance, it shows that the therapeutic effect of the antibody protein substance on the disease to which the target protein substance belongs is better. Therefore, the sample binding strength, obtained by using the server, between the sample antibody protein substance and the target protein substance may be used for issuing an excitation parameter or a penalty parameter to the initial prediction model to modify the model parameters of the initial prediction model, as described below.

The server may perform continuous model training on the initial prediction model for a plurality of times on the basis of the reference protein substance and the target protein substance, and perform model training on the initial prediction model once, and the initial prediction model may generate one sample predicted protein fragment, and then one sample binding strength is obtained. Therefore, when the model parameters of the initial prediction model are corrected on the basis of the sample binding strengths, the model parameters of the initial prediction model may be corrected on the basis of difference between the sample binding strengths obtained in the two-time adjacent model trainings to the initial prediction model.

Specifically, when the sample binding strength obtained by the next training for the initial prediction model is greater than the sample binding strength obtained by the previous training for the initial prediction model, it indicates that the accuracy of the sample predicted protein fragment obtained by prediction of the next training is higher than the accuracy of the sample predicted protein fragment obtained by prediction of the previous training, so that a reward parameter may be given to the initial prediction model in the next training process to encourage the initial prediction model to correct the model parameters in a good prediction direction (such as the prediction direction of the next time) on the basis of the reward parameter.

Conversely, when the sample binding strength obtained by the next training for the initial prediction model is less than the sample binding strength obtained by the previous training for the initial prediction model, it indicates that the accuracy of the sample predicted protein fragment obtained by prediction of the next training is lower than the accuracy of the sample predicted protein fragment obtained by prediction of the previous training, so that a penalty parameter may be given to the initial prediction model in the next training process, the initial prediction model corrects the model parameters in the good prediction direction (such as the previous prediction direction) instead of the poor prediction direction (such as the next prediction direction) according to the penalty parameter.

For example, in the nth training process of the initial prediction model, the server may synthesize a sample antibody protein substance kₙ by using the sample similar protein fragment matched with the sample predicted protein fragment generated by the initial prediction model, and the sample binding strength between the sample antibody protein substance kₙ and the target protein substance is the sample binding strength qₙ. n may be a positive integer less than or equal to the total number of training times for the initial prediction model.

In the (n−1) training process of the initial prediction model, the server may synthesize a sample antibody protein substance kₙ₋₁ by using a sample similar protein fragment matched with the sample predicted protein fragment generated by the initial prediction model, and the sample binding strength between the sample antibody protein substance kₙ₋₁ and the target protein substance may be sample binding strength qₙ₋₁.

Optionally, in the nth training process of the initial prediction model, the server may search, from the protein fragment database, a plurality of sample similar protein fragments matched with the sample predicted protein fragments, where the plurality of sample similar protein fragments may be N protein fragments that are most similar to the sample predicted protein fragments in the protein fragment database, and a specific value of N may be determined according to an actual application scenario, which is not limited thereto.

One sample similar protein fragment may correspond to one sample antibody protein substance kₙ. Therefore, a plurality (for example, N) of sample antibody protein substances kₙ exist, and each of the sample antibody protein substances kₙ has a binding strength (which may be referred to as a target binding strength) with the target protein substance, and therefore, an average value (which may be referred to as an average strength) of the target binding strength between each of all the sample antibody protein substances kₙ and the target protein substance may be used as the sample binding strength qₙ in the nth training process. Similarly, the sample binding strength qₙ₋₁ in the (n−1)th training process may be calculated by using a principle which is the same as a principle for calculating the sample binding strength qₙ.

Then, the server may correct the model parameters of the initial prediction model on the basis of the sample binding strength qₙ and the sample binding strength qₙ₋₁. For example, the server may acquire a squared difference between the sample binding strength qₙ and the sample binding strength qₙ₋₁, and the squared difference may be the value of the square of the sample binding strength qₙ minus the square of the sample binding strength qₙ₋₁.

The penalty parameter and the reward parameter above-mentioned may be collectively referred to as an excitation parameter, and the server may give the squared difference between the sample binding strength qₙ and the sample binding strength qₙ₋₁ above-mentioned to the initial prediction model as an excitation parameter, so that the initial prediction model corrects its model parameters on the basis of the excitation parameter.

It is understood that, if the sample binding strength qₙ is greater than the sample binding strength qₙ₋₁, the value of the squared difference between the sample binding strength qₙ and the sample binding strength qₙ₋₁ is a positive number, and at this time, the squared difference may be used as a reward parameter to the initial prediction model, so that the initial prediction model corrects its model parameters by using the reward parameter. If the sample binding strength qₙ is less than the sample binding strength qₙ₋₁, the value of the squared difference between the sample binding strength qₙ and the sample binding strength qₙ₋₁ is a negative number, and at this time, the squared difference may be used as a penalty parameter to the initial prediction model, so that the initial prediction model corrects its model parameters by using the penalty parameter.

By the above-mentioned process, the initial prediction model may be continuously trained by using the target protein substance and the reference protein substance, and when the initial prediction model converges or the training frequency of the initial prediction model reaches a certain threshold, the initial prediction model obtained by training at the moment may be used as the above-mentioned protein prediction model.

It is understood that, since each training of the initial prediction model corrects the model parameters of the initial prediction model, the sample predicted protein fragments obtained by prediction by using the initial prediction model in each training process are usually different, and correspondingly, the sample binding strength obtained in each training process is also usually different, and the model parameters of the initial prediction model may be continuously corrected on the basis of the different sample binding strengths obtained in each training process. This is equivalent to that sample data (which may be the sample binding strength in each training process) used for training the initial prediction model is generated by the initial prediction model itself, so that it is not necessary to prepare a large amount of sample data.

The sample binding strength between the sample antibody protein substance and the target protein substance may be obtained by performing protein-docking (an algorithm for calculating the interaction relationship between molecular substances) calculation on the sample antibody protein substance and the target protein substance.

From above, by the method provided by this embodiment of this application, the initial prediction model may be given the excitation parameter on the basis of the sample binding strength between the sample antibody protein substance synthesized by using the sample synthetic substance auxiliary information in an assisted manner and the target protein substance, so that the initial prediction model is subjected to reinforcement learning by using the excitation parameter, the final protein prediction model is obtained, and the protein prediction model may be configured to spontaneously predict the protein substance binding to the target protein substance on the basis of an AI technology.

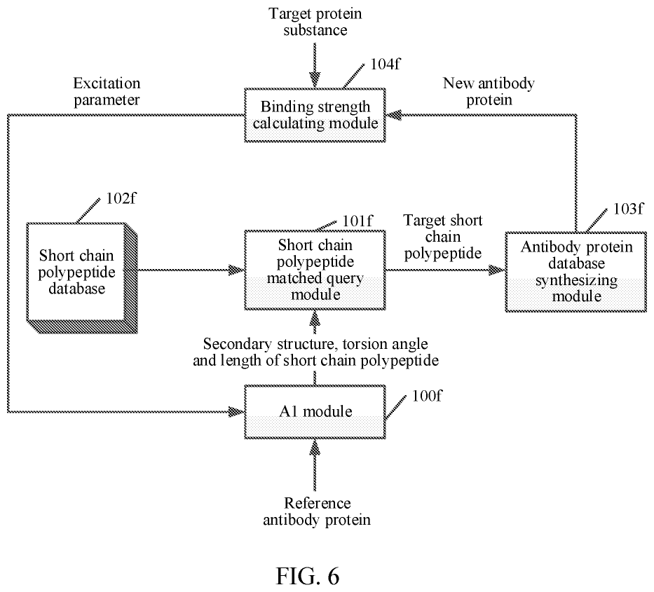

Referring to FIG. 6, FIG. 6 is a schematic diagram of a scenario of model training according to this application. As shown in FIG. 6, an AI module 100f may refer to the initial prediction model, and in this application, besides the AI module, a short chain polypeptide matched query module 101f, an antibody protein data synthesizing module 103f and a binding strength calculating module 104f are further included.

The server may input the protein attribute information of the reference antibody protein into the AI module, a secondary structure (namely the sample predicted structure information) and a torsion angle (namely the sample predicted torsion angle information) of the amino acid at the protein adjusting region may be predicted and obtained by the AI module.

On the basis of the length (namely the length of short chain polypeptide) of the original protein fragment at the protein adjusting region and the secondary structure and torsion angle of the amino acid at the protein adjusting region predicted and obtained by the AI module, a sample similar protein fragment may be matched from a short chain polypeptide database 102f, and the sample similar protein fragment is a target short chain polypeptide here.

The server may synthesize the target short chain polypeptide and the reference antibody protein by using the antibody protein data synthesizing module 103f to obtain a new antibody protein, and the new antibody protein is the sample antibody protein substance above-mentioned. Then, the server may calculate binding strength between the new antibody protein and the target protein substance by using the binding strength calculating module 104f, and an excitation parameter for the AI module may be generated by using the binding strength. The server may give the excitation parameter to the AI module to correct the model parameters of the AI module.

Through the above-mentioned process, the AI module 100f may be trained continuously and cyclically on the basis of the reference antibody protein and the target protein substance, and the trained AI module 100f is the above-mentioned protein prediction model.

Step S103: A similar protein fragment matching the predicted protein fragment is identified from the protein fragment database.

Specifically, the server may match a protein fragment similar to the predicted protein fragment predicted by the protein prediction model in the protein fragment database above-mentioned, and the matched protein fragment similar to the predicted protein fragment in the protein fragment database may be referred to as a similar protein fragment of the predicted protein fragment. The length of the similar protein fragment is the same as the length of the predicted protein fragment. The similar protein fragment of the predicted protein fragment may be matched from the protein fragment database by using a FragmentPicker tool (fragment selecting tool).

In this embodiment of this application, the data size, for matching the similar protein fragment, of the protein fragment database may be large, the predicted protein fragment obtained by prediction by using the protein prediction model provide the direction and basis of protein retrieval, and therefore, even if the number of protein fragments in the protein fragment database is extremely large, similar protein fragments having similar secondary structures and torsion angles may be quickly retrieved from the protein fragment database by the secondary structure (namely the predicted structure information above-mentioned) and the torsion angle (namely the predicted torsion angle information above-mentioned) of the predicted protein fragment.

Since the similar protein fragment is a protein fragment which is matched from the protein fragment database and is the most similar to the secondary structure and torsion angle of the predicted protein fragment, when the similar protein fragment of the predicted protein fragment is matched from the protein fragment database, one weight may be set for each of the predicted structure information and the predicted torsion angle information, the weight set for the predicted structure information may be referred to as a structure weight, and the weight set for the predicted torsion angle information may be referred to as a torsion angle weight. Therefore, the protein fragment having the highest overall similarity to the predicted structure information and the predicted torsion angle information may be matched from the protein fragment database according to the structure weight and the torsion angle weight as a similar protein fragment to the predicted protein fragment.

Step S104: Virtually synthesize the similar protein fragment and the reference protein substance to obtain synthetic substance auxiliary information; and the synthetic substance auxiliary information being configured to assist in generation of an antibody protein substance binding to the target protein substance.

Specifically, the server may virtually synthesize the matched similar protein fragment and the reference protein substance to obtain the synthetic substance auxiliary information. Virtual synthesis of the similar protein fragment and the reference protein substance may be performed by replacing the protein fragment at the protein adjusting region in the reference protein substance with the similar protein fragment. Virtual synthesis of the similar protein fragment and the reference protein substance refers to simulated synthesis of the similar protein fragment and the reference protein substance in a device instead of a real antibody protein substance synthesized by using a real similar protein fragment and the reference protein substance.

The synthetic substance auxiliary information may be related description information of a new protein substance obtained after synthesis of the similar protein substance and the reference protein substance (for example, description information such as protein structure information, protein sequence information and protein torsion angle information that can be used for describing and uniquely determining the protein substance). The synthetic substance auxiliary information is the related description information of the new protein substance obtained after replacing the protein fragment at the protein adjusting region in the reference protein substance with the similar protein fragment. The similar protein fragment and the reference protein substance may be virtually synthesized by a PyRosetta tool, where PyRosetta is a Rosetta (a table logic data tool based on a rough set theory framework) interaction suite based on Python (a computer programming language).

It is understood that, the synthetic substance auxiliary information above-mentioned is used for assisting in generation of an antibody protein substance binding to the target protein substance, the antibody protein substance is a protein substance obtained by replacing the protein fragment at the protein adjusting region in the reference protein substance with the similar protein fragment. The antibody protein substance is a macromolecular antibody protein which is generated on the basis of the similar protein fragment and is configured to bind to the target protein substance. The antibody protein substance is a finally predicted medicine for treating the disease to which the target protein substance belongs, and the antibody protein substance may bind to the target protein substance to realize treatment of the disease to which the target protein substance belongs.

For example, the server may cleave a protein fragment at the protein adjusting region in the reference protein substance to obtain a cleaved reference protein substance. The cleaved reference protein substance is a protein substance after the protein fragment at the protein adjusting region of the reference protein substance is removed. Then, the server may virtually synthesize the cleaved reference protein substance and the similar protein fragment to obtain synthetic substance auxiliary information. Virtual synthesis of the cleaved reference protein substance and the similar protein fragment may be performed by splicing the similar protein fragment to the cleaved reference protein substance at a position corresponding to the protein adjusting region.

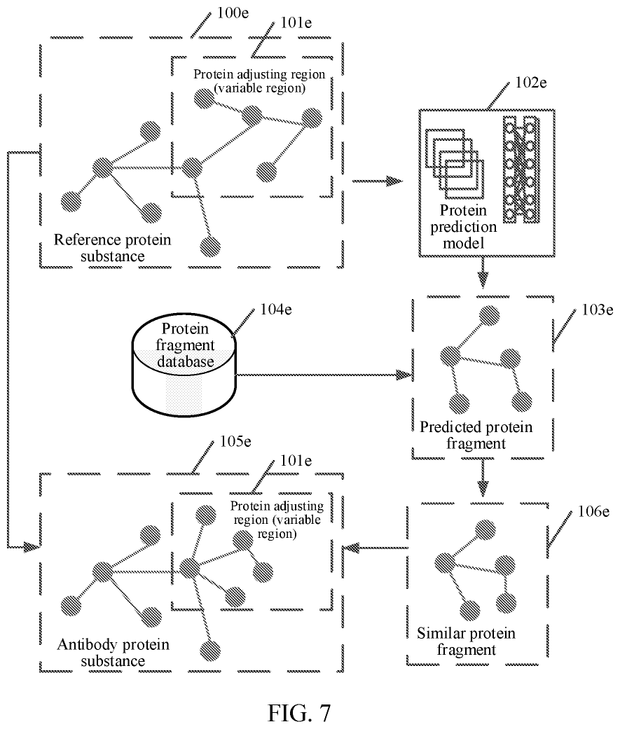

Referring to FIG. 7, FIG. 7 is a schematic diagram of a scenario of synthesis of an antibody protein substance according to this application. As shown in FIG. 7, a reference protein substance 100e may include a variable region 101e, and the variable region 101e is a protein adjusting region in the reference protein substance 100e.

The server may input the protein attribute information of the reference protein substance into a protein prediction model 102e, and correspondingly, the protein prediction model 102e may generate a predicted protein fragment 103e for a protein fragment at the protein adjusting region 101e. The fragment length of the predicted protein fragment 103e is the same as the fragment length of a protein fragment at the protein adjusting region 101e originally in the reference protein substance 100e.

Then, the server may match a similar protein fragment 106e of the predicted protein fragment 103e in a protein fragment database 104e. Then, the server may virtually synthesize the reference protein substance 100e and the similar protein fragment 106e to obtain synthetic substance auxiliary information. The synthetic substance auxiliary information may be related description information of antibody protein substances to be synthesized, and by the synthetic substance auxiliary information, a new antibody substance (namely the antibody protein substance 105e) obtained after synthesizing the reference protein substance 100e and the similar protein fragment 106e may be determined. As shown in FIG. 7, the antibody protein substance 105e is a protein substance obtained by replacing an original protein fragment at the protein adjusting region 101e in the reference protein substance 100e with the similar protein fragment.

Further, the server may generate a visual program file by using the synthetic substance auxiliary information, and the file format of the visual program file may be pdb (program data file) file format. By using the visual program file, substance visualization may be realized in the device for the antibody protein substance generated according to the synthetic substance auxiliary information, for example, by using the visual program file, a complete space picture of the antibody protein substance may be presented in the device, and the space picture may be a three-dimensional structure of the antibody protein substance.

Since the target protein substance may be provided by the pharmaceutical-factory-oriented third-party device, the server may transmit the generated visual program file of the synthetic substance auxiliary information to the third-party device. After the third-party device acquires the visual program file of the synthetic substance auxiliary information, the visual program file may be outputted in a device page, so that related researchers may research, develop, generate and improve the antibody protein substance according to the three-dimensional picture of the presented antibody protein substance. The target protein substance may be transmitted to the server in an on-line manner by the third-party device by using a related data transmitting technologies such as a cloud technology. Similarly, the server may predict and generate the synthetic substance auxiliary information above-mentioned in an on-line manner by using related technologies such as a cloud technology, and transmit the visual program file to the third-party device in an on-line manner by using related data transmitting technologies such as a cloud technology.

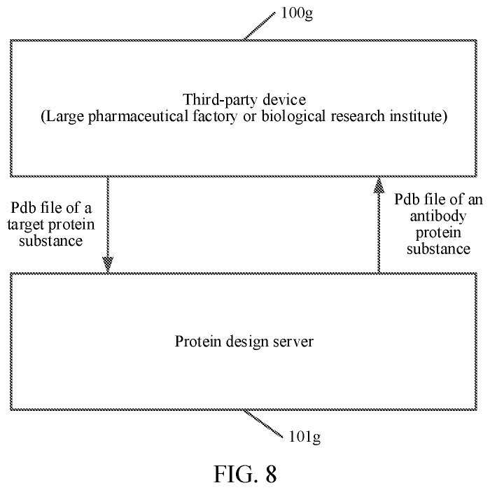

Referring to FIG. 8, FIG. 8 is a schematic diagram of a scenario of data interaction according to this application. The third-party device 100 g may be a device facing a large pharmaceutical factory or biological research institute. The third-party device 100 g may provide the pdb file of the target protein substance to a protein design server 101 g (namely the server that is the execution subject in this application). Further, the protein design server 101 g may generate the synthetic substance auxiliary information above-mentioned on the basis of the pdb file, provided by the third-party device, of the target protein substance, and may generate a pdb file for the antibody protein substance, that is, the visual program file above-mentioned, on the basis of the synthetic substance auxiliary information.

The protein design server 101 g may transmit the generated pdb file of the antibody protein substance to the third-party device 100 g, so that the third-party device 100 g may download the pdb file of the antibody protein substance for payment, and output the pdb file of the antibody protein substance in the device page after the download for payment, namely, the three-dimensional space picture of the antibody protein substance is presented, and the three-dimensional space picture is provided for relevant researchers to research, modify or develop the antibody protein substance.

In this application, the protein attribute information of the reference protein substance may be acquired; and the reference protein substance includes the protein adjusting region; Generating a predicted protein fragment at the protein adjusting region in the reference protein substance by using a protein prediction model according to the protein attribute information. The protein prediction model is obtained by training on the basis of the target protein substance, and the protein prediction model is configured to predict the protein substance binding to the target protein substance; the similar protein fragment of the predicted protein fragment is matched from the protein fragment database; the similar protein fragment and the reference protein substance are virtually synthesized to obtain synthetic substance auxiliary information; and the synthetic substance auxiliary information is configured to assist in generation of an antibody protein substance binding to the target protein substance. Therefore, according to the method provided by this application, the similar protein fragment configured to modify the protein fragment at the protein adjusting region in the reference protein substance is rapidly matched on the basis of the predicted protein fragment obtained by prediction by using the protein prediction model, the antibody protein substance configured to bind to the target protein substance may be rapidly generated on the basis of the similar protein fragment, and thus, the efficiency of acquiring the antibody protein substance is improved.

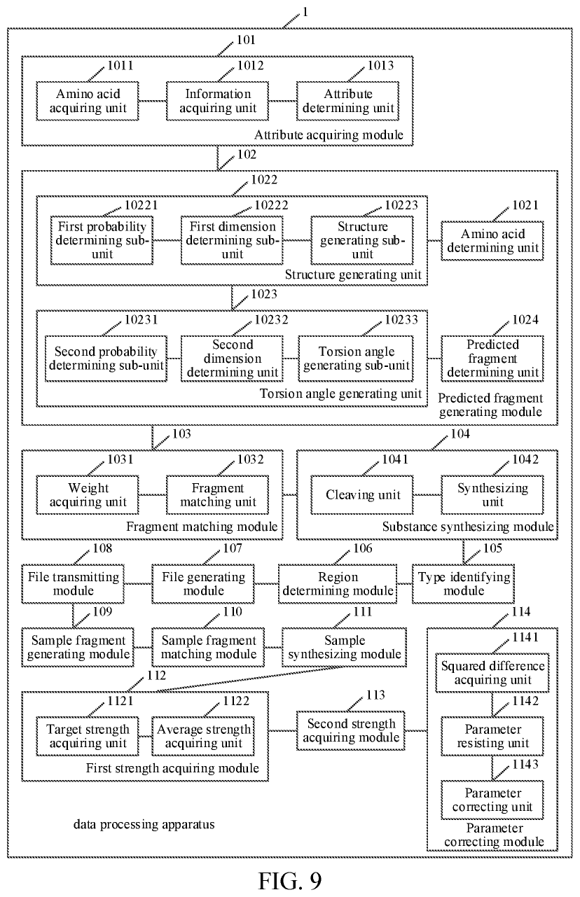

Referring to FIG. 9, FIG. 9 is a schematic structural diagram of a data processing apparatus according to this application. As shown in FIG. 9, the data processing apparatus 1 may include: an attribute acquiring module 101, a predicted fragment generating module 102, a fragment matching module 103 and a substance synthesizing module 104.

The attribute acquiring module 101 is configured to acquire the protein attribute information of the reference protein substance. The reference protein substance includes the protein adjusting region.

The predicted fragment generating module 102 is configured to generate a predicted protein fragment at the protein adjusting region in the reference protein substance by applying the protein prediction model to the protein attribute information. The protein prediction model being obtained on the basis of training of the target protein substance; and the protein prediction model is configured to predict a protein substance binding to the target protein substance.

The fragment matching module 103 is configured to match the similar protein fragment of the predicted protein fragment in the protein fragment database.

The substance synthesizing module 104 is configured to virtually synthesize the similar protein fragment and the reference protein substance to obtain the synthetic substance auxiliary information; and the synthetic substance auxiliary information is configured to assist in generation of an antibody protein substance binding to the target protein substance.

For a specific functional implementation of the attribute acquiring module 101, the predicted fragment generating module 102, the fragment matching module 103 and the substance synthesizing module 104, reference may be made to steps S101 to S104 in the embodiment corresponding to FIG. 3, and details are not described herein again.

The attribute acquiring module 101 includes: an amino acid acquiring unit 1011, an information acquiring unit 1012 and an attribute determining unit 1013.

The amino acid acquiring unit 1011 is configured to acquire at least two amino acids included by the reference protein substance.

The information acquiring unit 1012 is configured to acquire the amino acid structure information and the amino acid torsion angle information of each amino acid in at least two amino acids.

The attribute determining unit 1013 is configured to determine the amino acid structure information and the amino acid torsion angle information of each amino acid as the protein attribute information of the reference protein substance.

For a specific functional implementation of the amino acid acquiring unit 1011, the information acquiring unit 1012 and the attribute determining unit 1013, reference may be made to step S101 in the embodiment corresponding to FIG. 3, and details are not described herein again.

The predicted fragment generating module 102 includes: an amino acid determining unit 1021, a structure generating unit 1022, a torsion angle generating unit 1023 and a predicted fragment determining unit 1024.

The amino acid determining unit 1021 is configured to determine the amino acid at the protein adjusting region in the reference protein substance as an adjusted amino acid.

The structure generating unit 1022 is configured to generate predicted structure information corresponding to the adjusted amino acid by using the protein prediction model.

The torsion angle generating unit 1023 is configured to generate predicted torsion angle information corresponding to the adjusted amino acid by using the protein prediction model.

The predicted fragment determining unit 1024 is configured to determine the predicted protein fragment according to the predicted structure information and predicted torsion angle information corresponding to the adjusted amino acid.

For a specific functional implementation of the amino acid determining unit 1021, the structure generating unit 1022, the torsion angle generating unit 1023 and the predicted fragment determining unit 1024, reference may be made to step S102 in the embodiment corresponding to FIG. 3, and details are not described herein again.

The structure generating unit 1022 includes: a first probability determining sub-unit 10221, a first dimension determining sub-unit 10222 and a structure generating sub-unit 10223.

The first probability determining sub-unit 10221 is configured to determine the sampling probability of the adjusted amino acid on each amino acid structure dimension in at least two amino acid structure dimensions by using the protein prediction model.

The first dimension determining sub-unit 10222 is configured to determine the amino acid structure dimension with the maximum sampling probability in the at least two amino acid structure dimensions as a target structure dimension.

The structure generating sub-unit 10223 is configured to sample structure parameters on the target structure dimension to generate predicted structure information corresponding to the adjusted amino acid.

For a specific functional implementation of the first probability determining sub-unit 10221, the first dimension determining sub-unit 10222 and the structure generating sub-unit 10223, reference may be made to step S102 in the embodiment corresponding to FIG. 3, and details are not described herein again.

The torsion angle generating unit 1023 includes: a second probability determining sub-unit 10231, a second dimension determining unit 10232 and a torsion angle generating sub-unit 10233.

The second probability determining sub-unit 10231 is configured to determine the sampling probability of the adjusted amino acid on each amino acid torsion angle dimension in at least two amino acid torsion angle dimensions by using the protein prediction model.

The second dimension determining unit 10232 is configured to determine the amino acid torsion angle dimension with the maximum sampling probability in the at least two amino acid torsion angle dimensions as a target torsion angle dimension.

The torsion angle generating sub-unit 10233 is configured to sample torsion angle parameters on the target torsion angle dimension and generate predicted torsion angle information corresponding to the adjusted amino acid.

For a specific functional implementation of the second probability determining sub-unit 10231, the second dimension determining unit 10232 and the torsion angle generating sub-unit 10233, reference may be made to step S102 in the embodiment corresponding to FIG. 3, and details are not described herein again.

The fragment matching module 103 includes: a weight acquiring unit 1031 and a fragment matching unit 1032.

The weight acquiring unit 1031 is configured to acquire structure weight for the predicted structure information, and acquire torsion angle weight for the predicted torsion angle information.

The fragment matching unit 1032 is configured to match a similar protein fragment of the predicted protein fragment from the protein fragment database according to the structure weight, the torsion angle weight, the predicted structure information and the predicted torsion angle information.

For a specific functional implementation of the weight acquiring unit 1031 and the fragment matching unit 1032, reference may be made to step S103 in the embodiment corresponding to FIG. 3, and details are not described herein again.

The substance synthesizing module 104 includes: a cleaving unit 1041 and a synthesizing unit 1042.

The cleaving unit 1041 is configured to cleave the protein fragment at the protein adjusting region in the reference protein substance to obtain cleaved reference protein substance.

The synthesizing unit 1042 is configured to virtually synthesize the cleaved reference protein substance and the similar protein fragment to obtain synthetic substance auxiliary information.

For a specific functional implementation of the cleaving unit 1041 and the synthesizing unit 1042, reference may be made to step S104 in the embodiment corresponding to FIG. 3, and details are not described herein again.

The data processing apparatus 1 above-mentioned further includes: a type identifying module 105 and a region determining module 106.

The type identifying module 105 is configured to identify a target protein type of the target protein substance.

The region determining module 106 is configured to determine the protein adjusting region in the reference protein substance according to the target protein type.

For a specific functional implementation of the type identifying module 105 and the region determining module 106, reference may be made to step S101 in the embodiment corresponding to FIG. 3, and details are not described herein again.

The target protein substance is provided by the third-party device.

The data processing apparatus 1 above-mentioned further includes: a file generating module 107 and a file transmitting module 108.

The file generating module 107 is configured to generate a visual program file of the synthetic substance auxiliary information.

The file transmitting module 108 is configured to transmit the visual program file to the third-party device, so that the third-party device outputs the visual program file.

For a specific functional implementation of the file generating module 107 and the file transmitting module 108, reference may be made to step S104 in the embodiment corresponding to FIG. 3, and details are not described herein again.

The data processing apparatus 1 above-mentioned further includes: a sample fragment generating module 109, a sample fragment matching module 110, a sample synthesizing module 111, a first strength acquiring module 112, a second strength acquiring module 113 and a parameter correcting module 114.

The sample fragment generating module 109 is configured to input the protein attribute information into the initial prediction model, and generate the sample predicted protein fragment at the protein adjusting region in a nth training process of the initial prediction model, where n is an integer greater than 1.

The sample fragment matching module 110 is configured to match the sample similar protein fragment of the sample predicted protein fragment in the protein fragment database.

The sample synthesizing module 111 is configured to virtually synthesize the reference protein substance and the sample similar protein fragment to obtain the sample synthetic substance auxiliary information. The sample synthetic substance auxiliary information is used for assisting in generation of a sample antibody protein substance kₙ binding to the target protein substance.

The first strength acquiring module 112 is configured to acquire sample binding strength qₙ between the sample antibody protein substance kₙ and the target protein substance.

The second strength acquiring module 113 is configured to acquire sample binding strength qₙ₋₁ between the sample antibody protein substance kₙ₋₁ for the target protein substance and the target protein substance in a (n−1)th training process of the initial prediction model.

The parameter correcting module 114 is configured to correct model parameters of the initial prediction model according to the sample binding strength qₙ and the sample binding strength qₙ₋₁ to obtain the protein prediction model.

For a specific functional implementation of the sample fragment generating module 109, the sample fragment matching module 110, the sample synthesizing module 111, the first strength acquiring module 112, the second strength acquiring module 113 and the parameter correcting module 114, reference may be made to step S102 in the embodiment corresponding to FIG. 3, and details are not described herein again.

The number of the sample similar protein fragments is at least two. One sample similar protein fragment corresponds to one sample antibody protein substance kₙ.

The first strength acquiring module 112 includes: a target strength acquiring unit 1121 and an average strength acquiring unit 1122.

The target strength acquiring unit 1121 is configured to acquire the target binding strength between each of the at least two sample antibody protein substances kₙ and the target protein substance.

The average strength acquiring unit 1122 is configured to determine the average strength of target binding strengths corresponding to the at least two sample antibody protein substances kₙ as the sample binding strength qₙ.

For a specific functional implementation of the target strength acquiring unit 1121 and the average strength acquiring unit 1122, reference may be made to step S102 in the embodiment corresponding to FIG. 3, and details are not described herein again.

The parameter correcting module 114 includes: a squared difference acquiring unit 1141, a parameter resisting unit 1142 and a parameter correcting unit 1143.

The squared difference acquiring unit 1141 is configured to acquire squared difference between the sample binding strength qₙ and the sample binding strength qₙ₋₁.

The parameter resisting unit 1142 is configured to determine an excitation parameter for the initial prediction model according to the squared difference.

The parameter correcting unit 1143 is configured to correct the model parameters of the initial prediction model according to the excitation parameter to obtain the protein prediction model.

For a specific functional implementation of the squared difference acquiring unit 1141, the parameter resisting unit 1142 and the parameter correcting unit 1143, reference may be made to step S102 in the embodiment corresponding to FIG. 3, and details are not described herein again.

In this application, the protein attribute information of the reference protein substance may be acquired; and The reference protein substance includes the protein adjusting region. The predicted protein fragment at the protein adjusting region of the reference protein substance is generated by applying the protein prediction model to the protein attribute information. The protein prediction model being obtained on the basis of training of the target protein substance; and the protein prediction model is configured to predict a protein substance binding to the target protein substance. The similar protein fragment of the predicted protein fragment is matched from the protein fragment database. The similar protein fragment and the reference protein substance are virtually synthesized to obtain synthetic substance auxiliary information; and the synthetic substance auxiliary information is configured to assist in generation of an antibody protein substance binding to the target protein substance. Therefore, by the apparatus provided by this application, the similar protein fragment configured to modify the protein fragment at the protein adjusting region in the reference protein substance may be rapidly matched on the basis of the predicted protein fragment obtained by prediction by using the protein prediction model, then the antibody protein substance configured to bind to the target protein substance may be rapidly generated on the basis of the similar protein fragment, and thus, the efficiency of acquiring the antibody protein substance is improved.

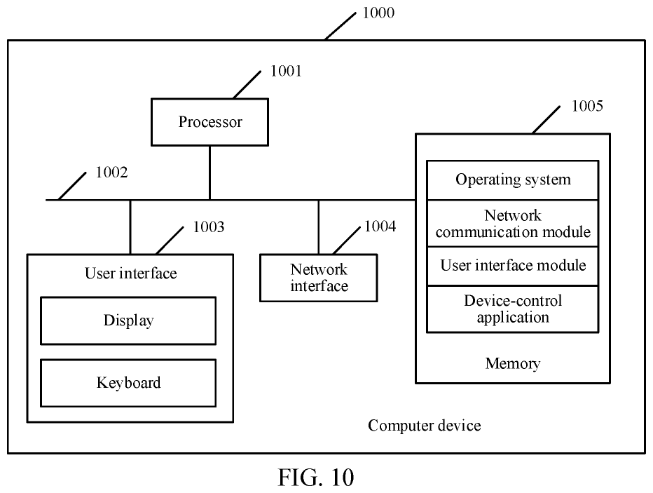

Referring to FIG. 10, FIG. 10 is a schematic structural diagram of a computer device according to this application. As shown in FIG. 10, a computer device 1000 may include: a processor 1001, a network interface 1004, and a memory 1005. In addition, the computer device 1000 may further include: a user interface 1003 and at least one communication bus 1002. The communication bus 1002 is configured to implement connection and communication between the components. The user interface 1003 may include a display, a keyboard, and in some embodiments, the user interface 1003 may further include a standard wired interface and a standard wireless interface. Optionally, the network interface 1004 may include a standard wired interface and a standard wireless interface (such as a Wi-Fi interface). The memory 1005 may be a high-speed RAM memory, or may be a non-volatile memory, for example, at least one magnetic disk memory. Optionally, the memory 1005 may be further at least one storage apparatus away from the foregoing processor 1001. As shown in FIG. 10, the memory 1005, which is used as a computer storage medium, may include an operating system, a network communication module, a user interface module, and a device control application program.

In the computer device 1000 shown in FIG. 10, the network interface 1004 may provide a network communication function; the user interface 1003 is mainly configured to provide an input interface for a user; and the processor 1001 may be configured to invoke the device-control application stored in the memory 1005, to implement the description of the data processing method according to the corresponding embodiments in the foregoing FIG. 3. It is to be understood that the computer device 1000 described in this application can implement the descriptions of the data processing apparatus 1 in the foregoing embodiment corresponding to FIG. 9. Details are not described herein again. In addition, the description of beneficial effects of the same method is not described herein again.

In addition, the embodiments of this application further provide a computer-readable storage medium. The computer-readable storage medium stores a computer program executed by the data processing apparatus 1 mentioned above, and the computer program includes program instructions. When executing the program instructions, the processor can perform the descriptions of data processing method in the embodiment corresponding to FIG. 3. Therefore, details are not described herein again. In addition, the description of beneficial effects of the same method is not described herein again. For technical details that are not disclosed in the computer storage medium embodiments of this application, refer to the descriptions of the method embodiments of this application.

A person of ordinary skill in the art may understand that all or some of the processes of the methods in the embodiments may be implemented by a computer program instructing relevant hardware. The program may be stored in a computer-readable storage medium. When the program is executed, the procedures of the foregoing method embodiments are performed. The foregoing storage medium may include a magnetic disc, an optical disc, a read-only memory (ROM), a random access memory (RAM), or the like.

What is disclosed above is merely exemplary embodiments of this application, and certainly is not intended to limit the scope of the claims of this application. Therefore, equivalent variations made in accordance with the claims of this application shall fall within the scope of this application.

What is claimed is:

1. A method for processing bioinformatic data performed by a computer device, the method comprising:
acquiring protein attribute information of a reference protein substance, the reference protein substance comprising a protein adjusting region;
generating a predicted protein fragment at the protein adjusting region in the reference protein substance by applying a protein prediction model to the protein attribute information, the protein prediction model being configured to predict a protein substance binding to a target protein substance;
identifying a similar protein fragment matching the predicted protein fragment in a protein fragment database; and
virtually synthesizing the similar protein fragment and the reference protein substance to obtain synthetic substance auxiliary information; and the synthetic substance auxiliary information being configured to assist in generation of an antibody protein substance that binds to the target protein substance.

2. The method according to claim 1, wherein the acquiring the protein attribute information of the reference protein substance comprises:
acquiring at least two amino acids in the reference protein substance;
acquiring amino acid structure information and amino acid torsion angle information of each amino acid in the at least two amino acids; and
determining the amino acid structure information and the amino acid torsion angle information of each amino acid into the protein attribute information of the reference protein substance.

3. The method according to claim 2, wherein the generating a predicted protein fragment at the protein adjusting region in the reference protein substance by applying a protein prediction model to the protein attribute information comprises:
determining an amino acid at the protein adjusting region in the reference protein substance as an adjusted amino acid;
generating, by using the protein prediction model, predicted structure information corresponding to the adjusted amino acid;
generating, by using the protein prediction model, predicted torsion angle information corresponding to the adjusted amino acid; and
determining the predicted protein fragment according to the predicted structure information and the predicted torsion angle information corresponding to the adjusted amino acid.

4. The method according to claim 1, wherein the virtually synthesizing the similar protein fragment and the reference protein substance to obtain the synthetic substance auxiliary information comprises:
cleaving a protein fragment at the protein adjusting region in the reference protein substance to obtain a cleaved reference protein substance; and
virtually synthesizing the cleaved reference protein substance and the similar protein fragment to obtain the synthetic substance auxiliary information.

5. The method according to claim 1, further comprising:
identifying a target protein type of the target protein substance; and
determining the protein adjusting region in the reference protein substance according to the target protein type.

6. A computer device, comprising a memory and a processor, the memory storing a computer program, the computer program, when executed by the processor, causing the computer device to perform a method for processing bioinformatic data including:
acquiring protein attribute information of a reference protein substance, the reference protein substance comprising a protein adjusting region;
generating a predicted protein fragment at the protein adjusting region in the reference protein substance by applying a protein prediction model to the protein attribute information, the protein prediction model being configured to predict a protein substance binding to a target protein substance;
identifying a similar protein fragment matching the predicted protein fragment in a protein fragment database;
virtually synthesizing the similar protein fragment and the reference protein substance to obtain synthetic substance auxiliary information; and the synthetic substance auxiliary information being configured to assist in generation of an antibody protein substance that binds to the target protein substance.

7. The computer device according to claim 6, wherein the acquiring the protein attribute information of the reference protein substance comprises:
acquiring at least two amino acids in the reference protein substance;
acquiring amino acid structure information and amino acid torsion angle information of each amino acid in the at least two amino acids; and
determining the amino acid structure information and the amino acid torsion angle information of each amino acid into the protein attribute information of the reference protein substance.

8. The computer device according to claim 7, wherein the generating a predicted protein fragment at the protein adjusting region in the reference protein substance by applying a protein prediction model to the protein attribute information comprises:
determining an amino acid at the protein adjusting region in the reference protein substance as an adjusted amino acid;
generating, by using the protein prediction model, predicted structure information corresponding to the adjusted amino acid;
generating, by using the protein prediction model, predicted torsion angle information corresponding to the adjusted amino acid; and
determining the predicted protein fragment according to the predicted structure information and the predicted torsion angle information corresponding to the adjusted amino acid.

9. The computer device according to claim 6, wherein the virtually synthesizing the similar protein fragment and the reference protein substance to obtain the synthetic substance auxiliary information comprises:
cleaving a protein fragment at the protein adjusting region in the reference protein substance to obtain a cleaved reference protein substance; and
virtually synthesizing the cleaved reference protein substance and the similar protein fragment to obtain the synthetic substance auxiliary information.

10. The computer device according to claim 6, wherein the method further comprises:
identifying a target protein type of the target protein substance; and
determining the protein adjusting region in the reference protein substance according to the target protein type.

11. A non-transitory computer-readable storage medium storing a computer program, and the computer program being adapted to be loaded and executed by a processor of a computer device and causing the computer device to implement a method for processing bioinformatic data including:
acquiring protein attribute information of a reference protein substance, the reference protein substance comprising a protein adjusting region;
generating a predicted protein fragment at the protein adjusting region in the reference protein substance by applying a protein prediction model to the protein attribute information, the protein prediction model being configured to predict a protein substance binding to a target protein substance;
identifying a similar protein fragment matching the predicted protein fragment in a protein fragment database;
virtually synthesizing the similar protein fragment and the reference protein substance to obtain synthetic substance auxiliary information; and the synthetic substance auxiliary information being configured to assist in generation of an antibody protein substance that binds to the target protein substance.

12. The non-transitory computer-readable storage medium according to claim 11, wherein the acquiring the protein attribute information of the reference protein substance comprises:
acquiring at least two amino acids in the reference protein substance;
acquiring amino acid structure information and amino acid torsion angle information of each amino acid in the at least two amino acids; and
determining the amino acid structure information and the amino acid torsion angle information of each amino acid into the protein attribute information of the reference protein substance.

13. The non-transitory computer-readable storage medium according to claim 11, wherein the generating a predicted protein fragment at the protein adjusting region in the reference protein substance by applying a protein prediction model to the protein attribute information comprises:
determining an amino acid at the protein adjusting region in the reference protein substance as an adjusted amino acid;
generating, by using the protein prediction model, predicted structure information corresponding to the adjusted amino acid;
generating, by using the protein prediction model, predicted torsion angle information corresponding to the adjusted amino acid; and
determining the predicted protein fragment according to the predicted structure information and the predicted torsion angle information corresponding to the adjusted amino acid.

14. The non-transitory computer-readable storage medium according to claim 11, wherein the virtually synthesizing the similar protein fragment and the reference protein substance to obtain the synthetic substance auxiliary information comprises:
cleaving a protein fragment at the protein adjusting region in the reference protein substance to obtain a cleaved reference protein substance; and
virtually synthesizing the cleaved reference protein substance and the similar protein fragment to obtain the synthetic substance auxiliary information.

15. The non-transitory computer-readable storage medium according to claim 11, wherein the method further comprises:
identifying a target protein type of the target protein substance; and
determining the protein adjusting region in the reference protein substance according to the target protein type.
